(19) United States
(12) Patent Application Publication
(10) Pub. No.: US 2026/0165751 A1
(43) Pub. Date: Jun. 18, 2026

(54) SCREW IMPLANT AND METHODS FOR SAME

(71) Applicant: Virgilio Matheus Rojas, Gotha, FL (US)

(72) Inventor: Virgilio Matheus Rojas, Gotha, FL (US)

(21) Appl. No.: 19/368,846

(22) Filed: Oct. 24, 2025

Related U.S. Application Data
(60) Provisional application No. 63/715,507, filed on Nov. 1, 2024.

(51) Int. Cl. A61B 17/70

(52) U.S. Cl. CPC A61B 17/7071

(57) ABSTRACT

A pedicle-lengthening implant may comprise an outer screw body configured to be inserted through a pedicle of a vertebra. The outer body has an upper portion and a lower portion. An inner bolt is disposed within the outer screw body, defining a longitudinal axis and configured to interact with each of the upper and the lower portions. Rotation of the inner bolt about the longitudinal axis causes the upper and lower portions of the outer screw body to separate axially along the longitudinal axis. The outer body may comprise external threads configured to engage bone of the pedicle. The inner bolt includes a threaded rod that engages first internal threads of the upper portion and internal threads of the lower portion at an inner bore of the outer screw body. The first internal threads of the upper portion and internal threads of the lower portion may have opposite thread directions.

### RELATED APPLICATION

Under provisions of 35 U.S.C. § 119(e), the Applicant claims benefit of U.S. Provisional Application No. 63/715,507 filed on Nov. 1, 2024, and having inventors in common, which is incorporated herein by reference in its entirety.

It is intended that the referenced application may be applicable to the concepts and embodiments disclosed herein, even if such concepts and embodiments are disclosed in the referenced application with different limitations and configurations and described using different examples and terminology.

### FIELD OF DISCLOSURE

The present disclosure generally relates to spinal surgical implants and instruments for treating spinal stenosis and related conditions. More specifically, the disclosure pertains to expandable screw implant systems and surgical methods for performing thoracolumbar laminoplasty procedures to decompress the spinal canal while preserving laminar structure.

### BACKGROUND

In some situations, spinal stenosis causes compression of neural elements within the spinal canal, leading to neurological symptoms and functional impairment. For example, degenerative changes in the thoracolumbar spine can result in narrowing of the central canal, causing compression of the spinal cord and nerve roots. Thus, the conventional strategy is to perform laminectomy procedures that completely remove the lamina and other posterior spinal elements to decompress the neural structures. This often causes problems because the conventional strategy does not preserve the structural integrity of the spine and may lead to postoperative instability. For example, removal of the lamina eliminates important posterior stabilizing elements, potentially requiring additional fusion procedures to restore spinal stability.

Conventional spinal decompression systems rely primarily on complete bone removal techniques. Laminectomy procedures involve surgical excision of the entire lamina using cutting instruments and bone removal tools. These systems require extensive surgical exposure through large incisions to access the affected spinal levels. The surgical approach typically involves stripping of paraspinal muscles from their bony attachments and retraction of soft tissues. Bone removal is accomplished using rongeurs, osteotomes, and high-speed burrs to completely excise the lamina and ligamentum flavum.

Traditional decompression hardware includes various cutting and removal instruments designed for bone excision. Kerrison rongeurs provide precise bone removal in confined spaces around neural elements. Osteotomes allow controlled splitting and removal of bony structures. High-speed pneumatic and electric burrs enable rapid bone removal but generate heat and bone debris. These instruments are designed specifically for tissue removal rather than preservation or repositioning of anatomical structures.

Existing laminoplasty techniques have been developed primarily for cervical spine applications. Cervical laminoplasty systems utilize plate and screw constructs to maintain lamina elevation after creating hinge points. These systems require bilateral exposure and placement of multiple hardware components. The plates are positioned laterally and secured with screws to maintain the elevated position of the lamina. However, these cervical-specific designs are not suitable for thoracolumbar applications due to different anatomical constraints and biomechanical requirements.

The thoracolumbar spine presents unique anatomical challenges that limit the applicability of existing decompression techniques. The larger vertebral bodies and increased biomechanical loads in this region require different hardware configurations compared to cervical applications. The thoracic and lumbar laminae are thicker and more robust than cervical laminae. The pedicle anatomy varies significantly between thoracic and lumbar levels. These anatomical differences prevent direct adaptation of cervical laminoplasty systems to thoracolumbar applications.

Current expandable interbody devices are designed for disc space applications and do not address lamina elevation requirements. These devices expand within the intervertebral space to restore disc height and provide fusion support. The expansion mechanisms are optimized for radial expansion rather than axial lengthening. The structural requirements for interbody support differ substantially from those needed for lamina elevation. Interbody devices must withstand compressive loads while lamina elevation hardware must provide distraction forces.

Minimally invasive spinal surgery techniques have evolved to reduce surgical morbidity and improve patient outcomes. Percutaneous pedicle screw placement has become standard practice for many spinal conditions. These techniques utilize small incisions and specialized instrumentation to access spinal structures. However, current minimally invasive approaches do not provide adequate solutions for spinal decompression in cases of central canal stenosis. The available percutaneous tools are designed primarily for stabilization rather than decompression procedures.

Spinal stenosis in the thoracolumbar region often requires extensive surgical exposure for adequate decompression. The surgical approach typically involves midline incisions extending over multiple spinal levels. Muscle dissection and retraction are required to visualize the laminae and facet joints. This extensive exposure increases surgical time and postoperative pain. Blood loss is often significant due to the vascular nature of the surgical field. Recovery times are prolonged due to the extent of soft tissue trauma.

The biomechanical consequences of complete lamina removal extend beyond immediate structural instability. Loss of the posterior tension band alters load distribution across the motion segment. The remaining facet joints experience increased stress concentrations. Progressive degenerative changes may occur at adjacent levels due to altered spinal mechanics. These biomechanical alterations often necessitate concurrent or subsequent fusion procedures to prevent progressive deformity.

There remains a substantial gap in available hardware solutions for thoracolumbar laminoplasty procedures. Current spinal decompression techniques require surgeons to choose between highly invasive laminectomy procedures that completely remove posterior spinal elements or accept inadequate decompression in cases where complete bone removal is contraindicated. The thoracolumbar spine lacks dedicated hardware systems that can provide controlled lamina elevation while maintaining structural integrity. Existing expandable spinal implants are designed for interbody applications and cannot provide the axial distraction forces required for effective lamina elevation. The absence of specialized thoracolumbar laminoplasty hardware forces surgeons to rely on cervical spine techniques that are biomechanically unsuitable for the increased loads and different anatomical constraints of the thoracic and lumbar regions. This hardware gap limits treatment options for patients with thoracolumbar spinal stenosis and contributes to the continued reliance on more invasive surgical approaches that carry higher risks of postoperative complications and prolonged recovery periods.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

A pedicle-lengthening screw implant for expanding a spinal canal may comprise an outer screw body configured to be inserted through a pedicle of a vertebra, where the outer screw body may have an upper portion and a lower portion. The implant may further comprise an inner bolt disposed within the outer screw body, defining a longitudinal axis and configured to interact with each of the upper and the lower portions. Rotation of the inner bolt about the longitudinal axis may cause the upper and lower portions of the outer screw body to separate axially along the longitudinal axis.

A screw implant for spinal surgery may comprise a screw body configured for insertion into a vertebra, an internal mechanism within the screw body, and an actuator configured to operate the internal mechanism. Operation of the internal mechanism may cause the screw body to expand from a first length to a second length greater than the first length.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

FIG. 1A depicts a perspective view of the expandable screw implant in a collapsed configuration;

FIG. 1B illustrates a cross-sectional view of a partially-assembled screw implant assembly;

FIG. 1C illustrates an exploded view of the screw implant assembly;

FIG. 2A shows a cross-sectional view of the screw implant assembly with the inner bolt positioned within the outer screw body;

FIG. 2B illustrates a cross-sectional view of the screw implant assembly showing the inner bolt in an alternative position relative to the outer screw body;

FIG. 3A presents a perspective view of the assembled screw implant;

FIG. 3B shows an exploded perspective view of the screw implant assembly with individual components separated along a vertical axis;

FIG. 4A depicts a cross-sectional view of the screw implant assembly showing internal components in a first configuration;

FIG. 4B illustrates a cross-sectional view of the screw implant assembly showing internal components in a second configuration;

FIG. 5A shows a perspective view of the screw implant with the upper section and threaded shaft;

FIG. 5B presents an exploded perspective view of the screw implant;

FIG. 6A shows a cross-sectional view of the screw implant without internal expansion components;

FIG. 6B illustrates a cross-sectional view of the screw implant with an internal expansion component inserted;

FIG. 6C depicts a cross-sectional view of the screw implant with the internal expansion component in an engaged position;

FIG. 7A presents a perspective view of the screw implant assembly;

FIG. 7B shows a cross-sectional view of the screw implant assembly revealing internal components and structure;

FIG. 8 illustrates an exploded view of the screw implant system including installation tools and screw components;

FIG. 9 depicts a surgical tool for creating tissue channels, showing both schematic and photographic views;

FIG. 10 presents a flowchart illustrating the surgical method for implanting and operating the expandable screw implant; and

FIG. 11 shows an anatomical view of the screw implant installed within a vertebra, demonstrating the relationship between the implant and surrounding bone structure.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely to provide a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such a term to mean based on the contextual use of the term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶ 6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subject matter disclosed under the header.

The thoracolumbar laminoplasty screw system addresses several technical challenges encountered in current spinal decompression procedures. Spinal stenosis in the thoracolumbar region may result from various pathological conditions that narrow the central spinal canal. The narrowing may compress neural structures and cause neurological symptoms including pain, numbness, and motor dysfunction. Traditional surgical approaches may involve complete removal of the lamina through laminectomy procedures. However, laminectomy may compromise spinal stability and may require additional fusion procedures to maintain structural integrity.

Existing laminoplasty techniques may be limited primarily to cervical spine applications. The biomechanical forces and anatomical constraints in the thoracolumbar region may present different challenges compared to the cervical spine. Current hardware solutions for thoracolumbar decompression may not provide adequate means for controlled lamina elevation while preserving anatomical structures. The absence of specialized hardware for thoracolumbar laminoplasty may force surgeons to rely on more invasive procedures that may result in greater tissue trauma and longer recovery periods.

The expandable screw implant system may address these limitations by providing a minimally invasive approach to spinal canal expansion. The system may enable surgeons to achieve decompression through controlled lamina elevation rather than complete removal. This approach may preserve the structural integrity of the spine while providing adequate space for neural structures. The minimally invasive nature of the procedure may reduce blood loss, surgical time, and post-operative complications compared to traditional open laminectomy procedures.

In surgical scenarios involving multilevel spinal stenosis, the expandable screw system may allow for targeted decompression at specific levels without compromising adjacent segments. The system may be particularly beneficial in elderly patients who may not tolerate extensive surgical procedures. The controlled expansion mechanism may enable precise adjustment of the decompression based on individual patient anatomy and pathology. The system may also provide an alternative for patients who may not be candidates for fusion procedures due to medical comorbidities or bone quality concerns.

The primary use case of thoracolumbar laminoplasty represents a paradigm shift in spinal decompression surgery. The procedure may involve creating a controlled pathway through the lamina and pedicle into the vertebral body. The expandable screw may then be inserted through this pathway and expanded to elevate the lamina gradually. This elevation may increase the cross-sectional area of the spinal canal without removing bone tissue. The preserved lamina may maintain the posterior tension band of the spine and may contribute to long-term spinal stability.

The expandable screw implant system may represent a comprehensive solution for thoracolumbar laminoplasty procedures. The system may enable surgeons to achieve controlled spinal canal expansion through a minimally invasive approach that preserves anatomical integrity while providing therapeutic decompression.

The pedicle-lengthening screw implant may function as the primary therapeutic device within the system. The implant may comprise an outer screw body configured for insertion through the pedicle of a vertebra. The outer screw body may include an upper portion and a lower portion that may be configured to separate axially during expansion. An inner bolt may be disposed within the outer screw body and may define a longitudinal axis for controlled expansion.

The outer screw body may include external threads configured to engage bone tissue of the pedicle during insertion. The external threads may provide secure anchoring and may distribute mechanical loads across the vertebral structures. The inner bolt may comprise a threaded rod that may engage with internal threads of both the upper portion and the lower portion. The internal threading arrangement may enable controlled axial separation through rotational input.

In some embodiments, the internal threads of the upper portion and the lower portion may have opposite thread directions. The opposing thread configuration may enable simultaneous movement of both portions (e.g., either towards one another or away from one another) when the inner bolt is rotated about its longitudinal axis. The inner bolt may include an upper threaded portion with a first thread direction and a lower threaded portion with an opposite thread direction. An unthreaded middle portion may be positioned between the threaded upper and lower portions to facilitate alignment during assembly and operation.

The system may include a retaining ring configured to secure the position of the inner bolt and prevent unwanted rotation. The retaining ring may comprise external threads that may engage with second internal threads of the upper portion at an outer bore of the outer screw body. The locking mechanism may maintain the expanded configuration once the desired canal expansion may be achieved.

Multiple tool interfaces may be incorporated into the implant design to enable sequential surgical manipulation. The outer screw body may include a head having a first tool interface on a proximal end of the upper portion. The first tool interface may be configured to engage a first rotation tool for initial screw insertion. A second tool interface may be positioned on a proximal end of the inner bolt 106 and may be configured to engage a second rotation tool for expansion actuation. A third tool interface may be located on a proximal end of the retaining ring and may be configured to engage a third rotation tool for locking mechanism engagement.

The expandable screw implant may be constructed from biocompatible materials suitable for long-term implantation. The outer screw body and inner bolt may comprise biocompatible metals such as titanium, stainless steel, or titanium alloys. The material selection may ensure mechanical strength, corrosion resistance, and biocompatibility for permanent implantation.

In alternative embodiments, the system may include expandable flanges or blades connected to the lower portion of the outer screw body. The expandable flanges may be housed within the upper portion during insertion and may radially extend when the upper and lower portions separate during expansion. The radial extension of the flanges may provide additional mechanical advantage for lamina elevation and may engage with the inner surface of a pedicle cut to maintain the expanded configuration.

The screw implant may be configured to function as a standard pedicle screw when in the unexpanded state. The collapsed configuration may enable insertion through standard pedicle trajectories without requiring specialized surgical approaches. The expansion capability may be activated only when therapeutic decompression may be required based on intraoperative assessment.

The internal expansion mechanism may be reversible in certain embodiments to allow the screw body to return from the expanded length to the collapsed length. The reversibility feature may enable intraoperative adjustment of the expansion magnitude or may allow for implant removal if necessary. The controlled expansion and retraction capability may provide surgeons with precise control over the decompression process.

The system may include specialized instrumentation for creating the necessary anatomical preparations. A pedicle cutting tool may be configured to create circumferential cuts within the pedicle from within a cylindrical passage. The cutting tool may comprise flexible cutting elements that may conform to curved pathways within the pedicle anatomy. A pedicle drill may be used to create the initial cylindrical passage prior to use of the cutting tools.

Surgical planning may incorporate imaging guidance systems to determine optimal screw trajectory and expansion parameters. Fluoroscopic or computed tomographic imaging may be used to verify proper positioning and to monitor expansion progress during the procedure. The imaging feedback may enable real-time adjustment of the expansion magnitude based on anatomical response and therapeutic requirements.

The expandable screw system may be particularly beneficial in cases involving multilevel spinal stenosis where traditional surgical approaches may compromise spinal stability. The controlled expansion mechanism may enable targeted decompression at specific vertebral levels without affecting adjacent segments. The preservation of the lamina and posterior elements may maintain the natural biomechanics of the spine while providing adequate neural decompression.

Patient selection criteria may include individuals with central spinal stenosis who may not be suitable candidates for traditional laminectomy procedures. Elderly patients with medical comorbidities may benefit from the minimally invasive nature of the procedure. Patients with poor bone quality who may not tolerate extensive fusion procedures may be appropriate candidates for the expandable screw system.

The surgical technique may involve percutaneous or minimally invasive approaches to access the target vertebral level. The reduced tissue trauma associated with the procedure may result in decreased blood loss, shorter operative times, and faster recovery periods compared to traditional open surgical approaches. The preservation of anatomical structures may contribute to improved long-term outcomes and reduced complications.

Post-operative monitoring may include serial imaging studies to verify maintenance of the expanded configuration and to assess the degree of neural decompression achieved. The permanent nature of the expansion may provide sustained therapeutic benefit without requiring ongoing mechanical intervention. The biocompatible materials and secure locking mechanisms may ensure long-term stability of the expanded construct.

The expandable screw implant system may represent a paradigm shift in the surgical management of thoracolumbar spinal stenosis by providing a hardware solution that enables anatomical preservation while achieving therapeutic objectives. The controlled expansion capability may offer surgeons precise control over the decompression process and may enable individualized treatment approaches based on specific patient anatomy and pathology.

The present disclosure includes many aspects and features. Moreover, while many aspects and features relate to, and are described in, the context of an expandable screw implant, embodiments of the present disclosure are not limited to use only in this context.

### I. Platform Overview

This overview is provided to introduce a selection of concepts in a simplified form that are further described below. This overview is not intended to identify key features or essential features of the claimed subject matter. Nor is this overview intended to be used to limit the claimed subject matter's scope.

The expandable screw implant system may provide surgeons with a comprehensive solution for achieving spinal canal decompression while preserving anatomical integrity. The system may enable controlled expansion of the central canal through mechanical elevation of the lamina rather than complete removal of bone tissue. This approach may maintain the structural continuity of the spine while providing therapeutic relief from neural compression.

The expandable screw implant may function through a coordinated mechanism involving multiple components that work together to achieve controlled expansion. The outer screw body 102 may serve as the primary structural element that engages with vertebral bone tissue through external threading. The upper portion 104a and lower portion 104b of the outer screw body may be configured to separate axially when the internal expansion mechanism may be actuated. This separation may create the mechanical force necessary to elevate the lamina and expand the spinal canal diameter.

The inner bolt 106 may function as the actuating element that drives the expansion process. The inner bolt may comprise threaded portions that engage with corresponding internal threads within the upper and lower portions of the outer screw body. When the inner bolt may be rotated about its longitudinal axis, the threaded engagement may cause the upper and lower portions to move away from each other along the axis of the screw. This axial separation may translate into lamina elevation and subsequent canal expansion.

The threading configuration may be designed to provide mechanical advantage during the expansion process. The internal threads of the upper portion and lower portion may have opposite directions to enable simultaneous movement of both portions when the inner bolt may be rotated. The inner bolt may include an upper threaded portion with a first thread direction and a lower threaded portion with an opposite thread direction. An unthreaded middle portion may be positioned between the threaded sections to facilitate proper alignment during assembly and operation.

The retaining ring 110 may provide a locking mechanism to secure the expanded configuration once the desired canal enlargement may be achieved. The retaining ring may comprise external threads that engage with second internal threads of the upper portion at an outer bore of the outer screw body. This locking arrangement may prevent unwanted rotation of the inner bolt and may maintain the expanded position permanently.

The tool interface system may enable sequential surgical manipulation of the implant components. The outer screw body may include a head having a first tool interface on a proximal end of the upper portion. This first tool interface may be configured to engage a first rotation tool for initial screw insertion into the vertebral anatomy. A second tool interface may be positioned on a proximal end of the inner bolt and may be configured to engage a second rotation tool for expansion actuation. A third tool interface may be located on a proximal end of the retaining ring and may be configured to engage a third rotation tool for locking mechanism engagement.

The surgical procedure may begin with the creation of a pedicle passage through standard drilling techniques. The expandable screw may be inserted through this passage in its collapsed configuration, allowing for standard pedicle screw placement techniques. The screw may function as a conventional pedicle screw when in the unexpanded state, providing familiar handling characteristics for surgeons. Once proper positioning may be verified through imaging guidance, the expansion mechanism may be activated to achieve the desired canal enlargement.

The expansion process may be controlled and reversible in certain embodiments. The internal mechanism may allow the screw body to return from the expanded length to the collapsed length if adjustment may be required during the procedure. This reversibility feature may provide surgeons with precise control over the final expansion magnitude and may enable intraoperative fine-tuning based on patient response and anatomical considerations.

The biocompatible construction of the implant components may ensure long-term compatibility with human tissue. The outer screw body and inner bolt may comprise biocompatible metals such as titanium, stainless steel, or titanium alloys. These materials may provide the necessary mechanical strength for load-bearing applications while maintaining corrosion resistance and biocompatibility for permanent implantation.

In alternative embodiments, the system may include expandable flanges or blades connected to the lower portion of the outer screw body. These expandable elements may be housed within the upper portion during insertion and may extend radially when the upper and lower portions separate during expansion. The radial extension of these elements may provide additional mechanical advantage for lamina elevation and may engage with the inner surface of the pedicle to maintain the expanded configuration.

The expandable screw system may be particularly advantageous in multilevel spinal stenosis cases where traditional surgical approaches may compromise overall spinal stability. The controlled expansion capability may enable targeted decompression at specific vertebral levels without affecting adjacent segments. The preservation of the lamina and posterior spinal elements may maintain the natural biomechanics of the spine while providing adequate neural decompression.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

### II. Platform Configuration

The expandable screw implant system may comprise an integrated hardware solution designed to facilitate thoracolumbar laminoplasty procedures through controlled spinal canal expansion. The system may enable surgeons to achieve spinal decompression while preserving anatomical structures through a minimally invasive approach that may reduce surgical trauma compared to traditional laminectomy procedures.

The core components of the system may work in coordination to provide controlled expansion functionality. The outer screw body may serve as the primary structural element that interfaces with vertebral bone tissue through external threading. The upper portion and lower portion of the outer screw body may be configured to separate axially when the expansion mechanism may be activated. The inner bolt may function as the primary actuating component that drives the expansion process through threaded engagement with both portions of the outer screw body.

The expansion mechanism may operate through a coordinated threading system that enables controlled axial separation of the screw body portions. The inner bolt may comprise threaded sections with opposite thread directions that engage with corresponding internal threads within the upper and lower portions. When the inner bolt may be rotated about its longitudinal axis, the opposing thread configuration may cause simultaneous movement of both portions away from each other along the screw axis. This mechanical arrangement may provide the force necessary to elevate the lamina and expand the spinal canal diameter.

The retaining ring 110 may provide a secure locking mechanism to maintain the expanded configuration once the desired canal enlargement may be achieved. The retaining ring may comprise external threads that engage with internal threads of the upper portion at an outer bore of the outer screw body. This locking arrangement may prevent unwanted rotation of the inner bolt and may maintain the expanded position permanently throughout the healing process.

The tool interface system may enable sequential surgical manipulation through multiple access points. The outer screw body may include a head having a first tool interface positioned on a proximal end of the upper portion for initial screw insertion. A second tool interface may be located on a proximal end of the inner bolt for expansion actuation. A third tool interface may be positioned on a proximal end of the retaining ring for locking mechanism engagement. This multi-interface design may allow surgeons to perform insertion, expansion, and locking operations using specialized instrumentation.

The surgical procedure may begin with the creation of a pedicle passage using standard drilling techniques followed by circumferential pedicle cutting using specialized flexible cutting tools. The expandable screw may be inserted through this passage in its collapsed configuration, allowing for conventional pedicle screw placement techniques. Once proper positioning may be verified through imaging guidance, the expansion mechanism may be activated to achieve the desired degree of lamina elevation and canal expansion.

The system may include specialized instrumentation configured to create the necessary anatomical preparations for screw placement. A pedicle drill may be configured to create a cylindrical passage through the pedicle prior to screw insertion. The pedicle drill may comprise a cannulated drill bit that may be guided over a guidewire to ensure accurate trajectory through the pedicle anatomy. The drill may be available in multiple diameters to accommodate different screw sizes and patient anatomies.

A pedicle cutting tool may be configured to create circumferential cuts within the pedicle from within the cylindrical passage. The cutting tool may comprise flexible cutting elements that may conform to curved pathways within the pedicle anatomy. The flexible cutting elements may comprise nitinol wire or flexible metal strips that may be configured to cut through bone tissue when actuated. The pedicle cutting tool may include a twist handle 908 that may enable controlled rotation of the cutting elements to create precise circumferential osteotomies.

The pedicle cutting tool may include a bit elevation screw head that may enable vertical adjustment of the cutting bit position during the procedure. A cutting bit depth wheel may provide controlled depth adjustment to ensure precise cutting depth through the pedicle cortex. The combination of elevation and depth controls may enable surgeons to create customized osteotomy patterns based on individual patient anatomy and surgical requirements.

Surgical planning instrumentation may incorporate imaging guidance systems to determine optimal screw trajectory and expansion parameters. Fluoroscopic imaging may be used during the procedure to verify proper screw positioning and to monitor expansion progress in real-time. Computed tomographic imaging may be used for pre-operative planning to determine optimal entry points and screw trajectories based on patient-specific anatomy.

The expandable screw system may be configured for use with standard spinal surgery instrumentation where compatible. The screw head may be designed to interface with existing pedicle screw drivers and insertion instruments. The tool interfaces may be standardized to enable compatibility with commonly used surgical instrument sets. This compatibility may reduce the learning curve for surgeons and may minimize the need for specialized training on new instrumentation.

Post-operative imaging may be performed to verify maintenance of the expanded configuration and to assess the degree of neural decompression achieved. Serial radiographic studies may monitor the long-term stability of the expanded construct and may detect any potential complications such as screw loosening or hardware failure. The permanent nature of the expansion may provide sustained therapeutic benefit without requiring ongoing mechanical intervention or revision procedures.

The expandable screw implant system may be particularly advantageous in cases involving multilevel spinal stenosis where traditional surgical approaches may compromise overall spinal stability. The controlled expansion capability may enable targeted decompression at specific vertebral levels without affecting adjacent segments. The preservation of the lamina and posterior spinal elements may maintain the natural biomechanics of the spine while providing adequate neural decompression.

Patient selection criteria may include individuals with central spinal stenosis who may not be suitable candidates for traditional laminectomy procedures. Elderly patients with medical comorbidities may benefit from the minimally invasive nature of the procedure and the reduced surgical trauma associated with the expandable screw approach. Patients with poor bone quality who may not tolerate extensive fusion procedures may be appropriate candidates for the expandable screw system due to the preservation of anatomical structures and the reduced surgical invasiveness.

The surgical technique may involve percutaneous or minimally invasive approaches to access the target vertebral level. The reduced tissue trauma associated with the procedure may result in decreased blood loss, shorter operative times, and faster recovery periods compared to traditional open surgical approaches. The preservation of anatomical structures may contribute to improved long-term outcomes and reduced complications such as adjacent segment degeneration or spinal instability.

The expandable screw system may be configured to accommodate different expansion magnitudes based on the degree of stenosis and the amount of decompression required. The controlled expansion mechanism may enable incremental adjustment of the canal diameter during the procedure. Surgeons may be able to titrate the expansion based on intraoperative assessment of neural decompression and patient response.

The biocompatible construction of the implant components may ensure long-term compatibility with human tissue and may minimize the risk of adverse reactions or implant rejection. The materials used in the construction may be selected for their mechanical properties, corrosion resistance, and biocompatibility. Surface treatments or coatings may be applied to enhance osteointegration and to promote bone growth around the implant.

The expandable screw implant system may represent a paradigm shift in the surgical management of thoracolumbar spinal stenosis by providing a hardware solution that enables anatomical preservation while achieving therapeutic objectives. The controlled expansion capability may offer surgeons precise control over the decompression process and may enable individualized treatment approaches based on specific patient anatomy and pathology. The system may provide a valuable alternative to traditional surgical approaches and may expand treatment options for patients with spinal stenosis.

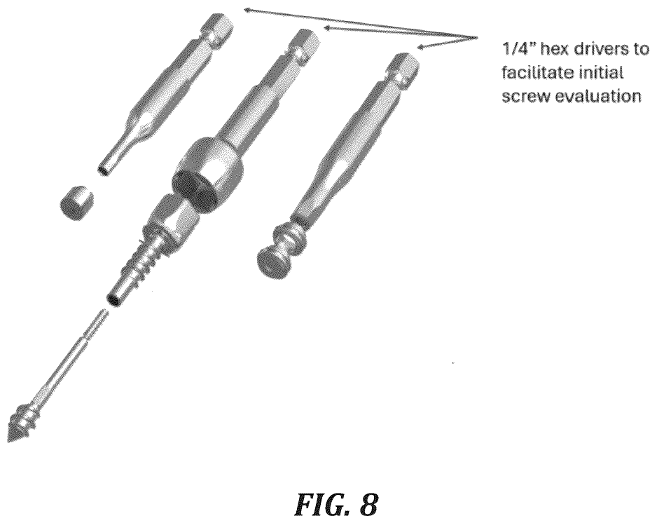

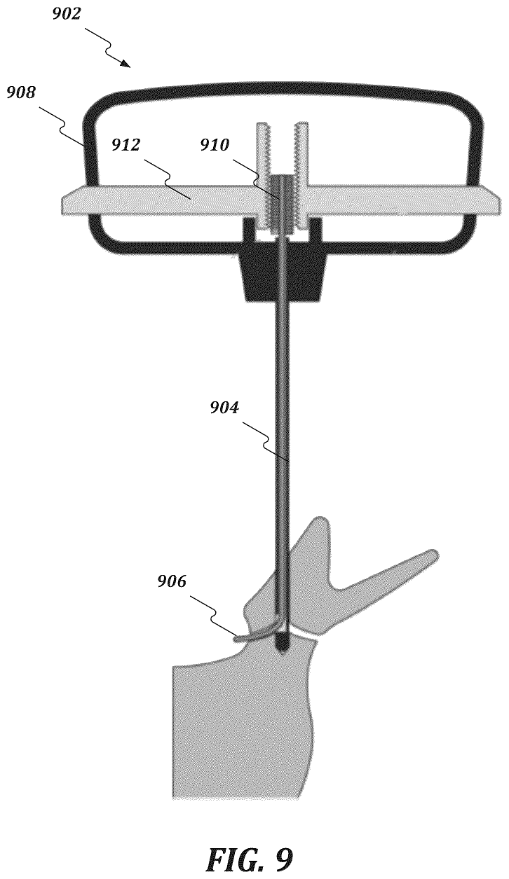

As best shown in FIGS. 8 and 9, the expandable screw system may include specialized instrumentation configured to create the necessary anatomical preparations for screw placement. A pedicle drill 902 may be configured to create a cylindrical passage through the pedicle prior to screw insertion. The pedicle drill may comprise a cannulated drill bit that may be guided over a guidewire to ensure accurate trajectory through the pedicle anatomy. The drill may be available in multiple diameters to accommodate different screw sizes and patient anatomies.

A pedicle cutting tool 904 may be configured to create circumferential cuts within the pedicle from within the cylindrical passage. The cutting tool may comprise flexible cutting elements 906 that may conform to curved pathways within the pedicle anatomy. The flexible cutting elements may comprise nitinol wire or flexible metal strips that may be configured to cut through bone tissue when actuated. The pedicle cutting tool may include a twist handle 908 that may enable controlled rotation of the cutting elements to create precise circumferential osteotomies.

The pedicle cutting tool may include a bit elevation screw head 910 that may enable vertical adjustment of the cutting bit position during the procedure. A cutting bit depth wheel 912 may provide controlled depth adjustment to ensure precise cutting depth through the pedicle cortex. The combination of elevation and depth controls may enable surgeons to create customized osteotomy patterns based on individual patient anatomy and surgical requirements.

Alternative embodiments of the expandable screw system may incorporate different expansion mechanisms to achieve controlled lamina elevation. In one embodiment, the system may include pneumatic expansion elements that may be inflated with saline or contrast medium to create controlled pressure within the vertebral anatomy. The pneumatic elements may comprise biocompatible balloon materials that may expand radially or axially when pressurized through a delivery catheter.

In another embodiment, the expansion mechanism may comprise shape memory alloy components that may change configuration when exposed to body temperature. The shape memory elements may be programmed to expand from a compressed delivery configuration to an expanded therapeutic configuration upon implantation. The transformation may be triggered by the thermal environment within the patient anatomy or through external heating devices.

The expandable screw system may include alternative threading configurations to optimize bone purchase and expansion control. In some embodiments, the external threads of the outer screw body may comprise variable pitch threading that may provide different mechanical advantages along the length of the screw. The variable pitch design may enable fine adjustment of expansion force distribution and may accommodate different bone densities encountered during insertion.

The internal threading arrangement may incorporate multiple thread starts to enable rapid expansion with fewer rotational inputs. A dual-start or triple-start thread configuration may reduce the number of turns required to achieve full expansion while maintaining mechanical advantage. The multi-start threading may also provide redundancy in the expansion mechanism to ensure reliable operation even if one thread path becomes compromised.

Alternative locking mechanisms may be incorporated to secure the expanded configuration of the screw system. In one embodiment, the locking mechanism may comprise a ratcheting system that may allow expansion in one direction while preventing retraction. The ratcheting elements may engage automatically during expansion and may require specific tool manipulation to disengage for revision procedures.

In another embodiment, the locking mechanism may comprise a cam-actuated system that may clamp the internal components in position through rotational input. The cam mechanism may provide positive locking with visual or tactile feedback to confirm engagement. The cam system may be reversible to allow controlled retraction of the expansion if intraoperative adjustment may be required.

The expandable screw system may include alternative tool interface configurations to accommodate different surgical preferences and instrument availability. In some embodiments, the tool interfaces may comprise standardized connections that may be compatible with existing spinal surgery instrument sets. The standardized interfaces may reduce the learning curve for surgeons and may minimize inventory requirements for surgical facilities.

Alternative tool interface designs may incorporate quick-connect mechanisms that may enable rapid engagement and disengagement during surgery. The quick-connect features may include bayonet-style connections, twist-lock mechanisms, or magnetic coupling systems. The rapid connection capability may reduce operative time and may improve surgical efficiency during multi-level procedures.

The expandable screw system may include alternative materials and surface treatments to enhance biocompatibility and osteointegration. In some embodiments, the screw components may comprise titanium alloys with specific surface texturing to promote bone ingrowth. The surface treatments may include hydroxyapatite coatings, plasma spray applications, or acid etching to create micro-roughened surfaces that may encourage cellular attachment.

Alternative material combinations may incorporate biodegradable polymers for specific components that may not require permanent implantation. The biodegradable elements may provide temporary structural support during the healing phase and may be resorbed by the body over time. The polymer components may be designed with controlled degradation rates to match the expected bone healing timeline.

The expandable screw system may include alternative expandable element configurations to optimize mechanical performance and anatomical fit. In some embodiments, the expandable elements may comprise articulated segments that may conform to irregular bone surfaces. The articulated design may provide better contact with the vertebral anatomy and may distribute loads more evenly across the bone-implant interface.

Alternative expandable element designs may incorporate flexible membranes or bellows structures that may expand uniformly in multiple directions. The flexible expansion elements may accommodate anatomical variations and may provide more predictable expansion characteristics compared to rigid mechanical systems. The membrane materials may be selected for durability and biocompatibility over the expected implant lifetime.

The expandable screw system may include alternative guidance and visualization systems to enhance surgical accuracy and safety. In some embodiments, the system may incorporate radiopaque markers at specific locations to enable real-time visualization during fluoroscopic guidance. The markers may be positioned to indicate screw orientation, expansion status, and anatomical landmarks during the procedure.

Alternative guidance systems may include integrated sensors that may provide feedback on insertion force, expansion pressure, or anatomical contact. The sensor feedback may be transmitted wirelessly to external monitoring equipment to provide real-time procedural data. The sensor information may enable surgeons to optimize screw placement and expansion parameters based on objective measurements rather than subjective assessment alone.

The expandable screw system may include alternative delivery and insertion techniques to accommodate different surgical approaches and patient anatomies. In some embodiments, the system may be configured for percutaneous insertion through minimal access techniques. The percutaneous approach may utilize specialized insertion guides and depth control mechanisms to ensure accurate placement without direct visualization of the target anatomy.

Alternative delivery techniques may incorporate robotic assistance systems that may provide enhanced precision and consistency during screw placement. The robotic systems may be programmed with patient-specific anatomical data to optimize insertion trajectories and expansion parameters. The robotic assistance may reduce variability between surgeons and may improve overall procedural outcomes through standardized techniques.

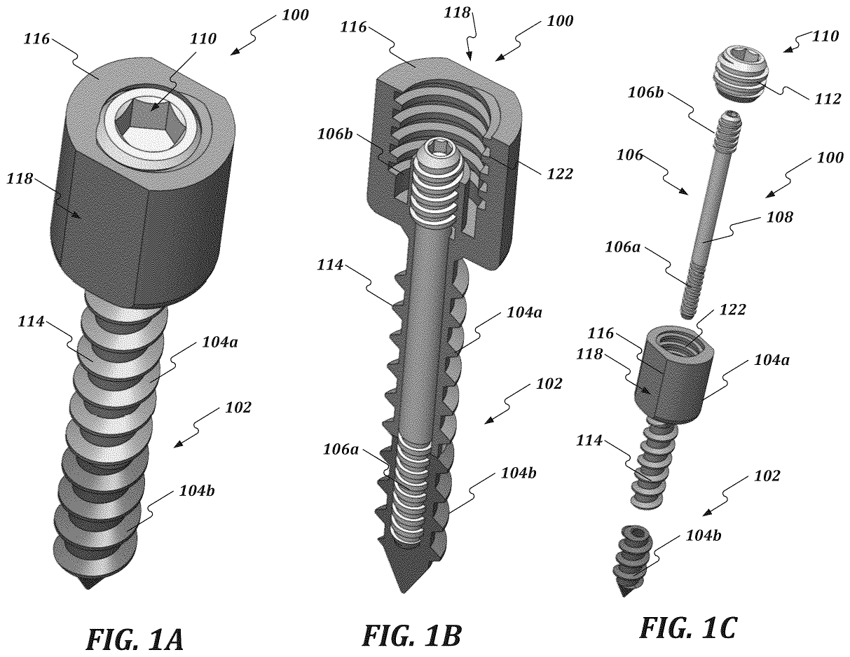

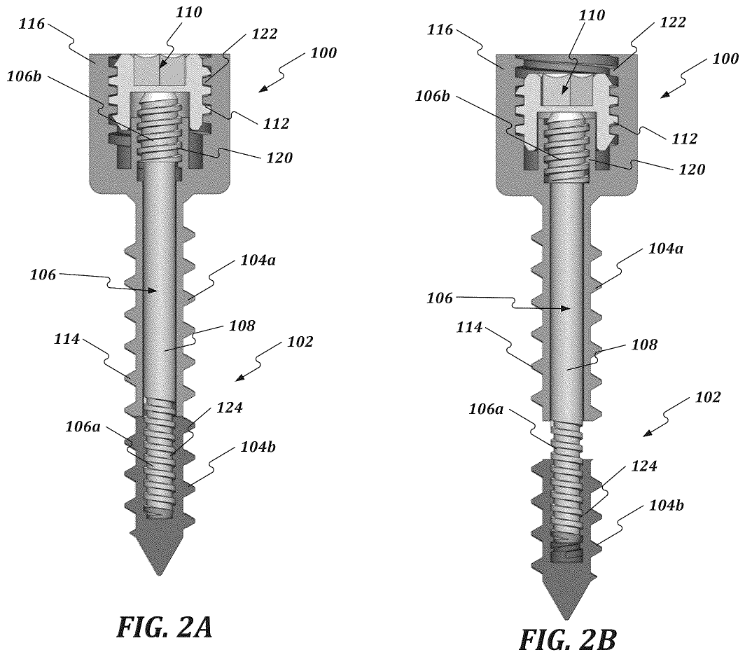

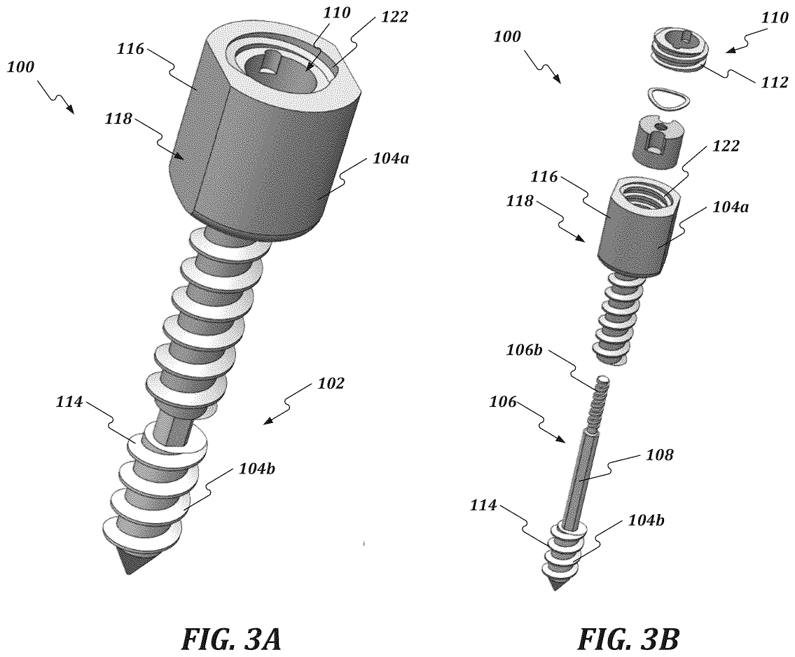

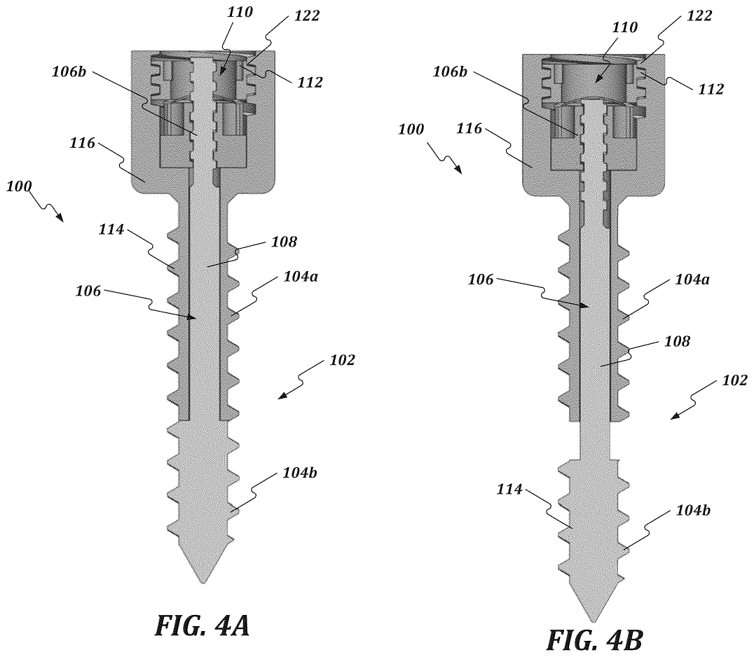

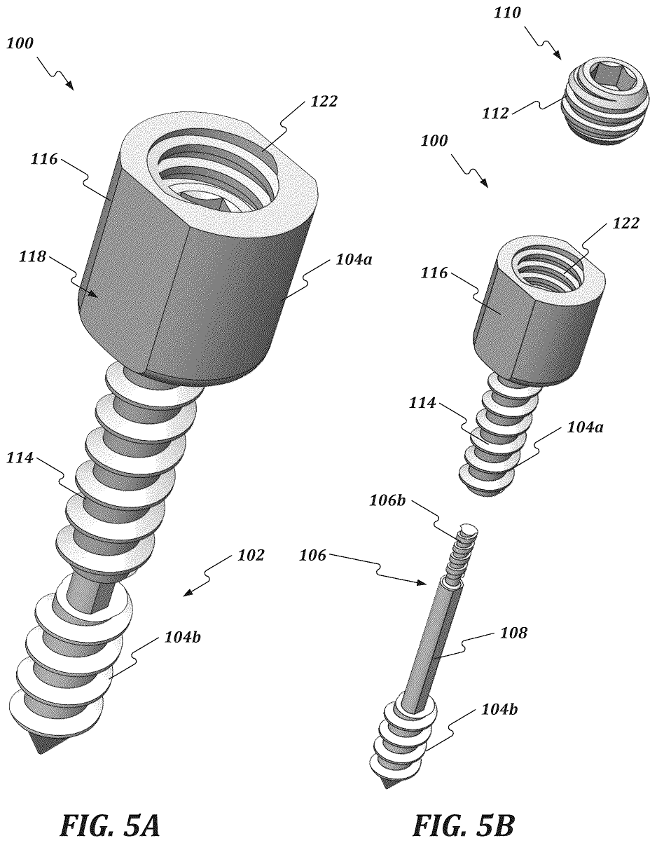

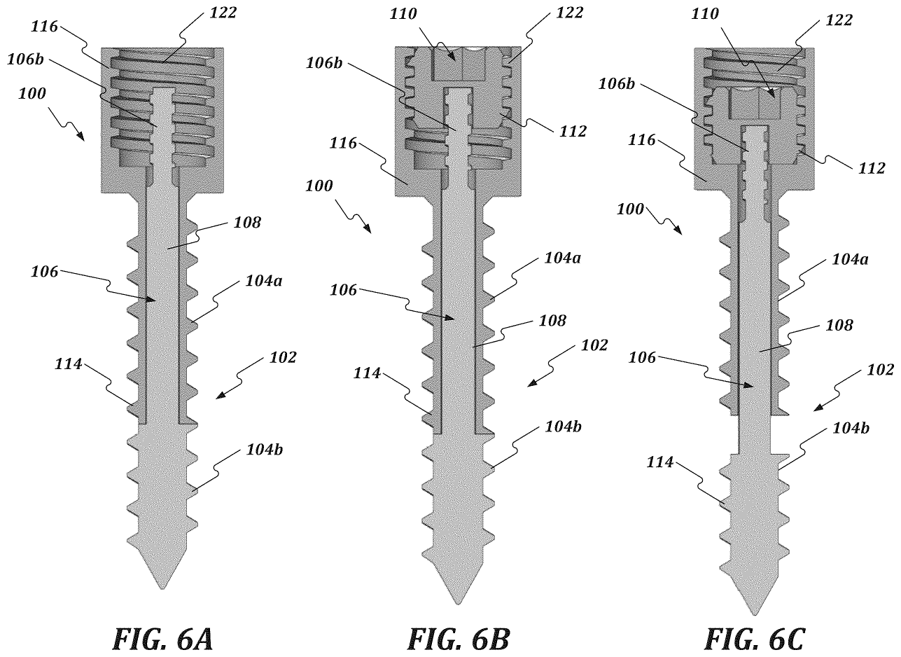

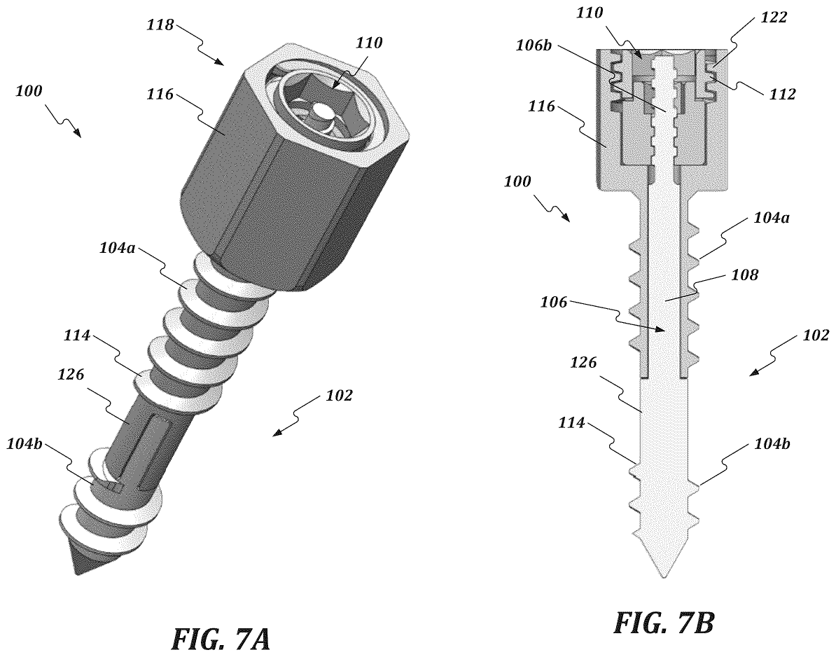

Accordingly, as best shown in FIGS. 1A-8, embodiments of the present disclosure provide a software and hardware platform comprised of a distributed set of computing elements, including, but not limited to:

### A. An Outer Screw Body

The screw implant device 100 may include an outer screw body 102. The outer screw body 102 may comprise a cannulated structure configured to accommodate the inner bolt 106 within its central bore. The outer screw body 102 may include external threads 114 disposed along at least a portion of its outer surface. The external threads 114 may be configured to engage with bone tissue of the pedicle during insertion and may provide secure anchoring within the vertebral anatomy.

The outer screw body 102 may comprise an upper (or proximal) portion 104a and a lower (or distal) portion 104b that may be configured to separate axially when the expansion mechanism is actuated. The upper portion 104a may include a head 116 positioned at its proximal end. The head 116 may comprise a first tool interface 118 configured to engage with a first rotation tool for initial screw insertion into the vertebral anatomy.

The upper portion 104a may include first internal threads 120 disposed within its inner bore. The first internal threads 120 may be configured to engage with corresponding threads on the inner bolt 106. The upper portion 104a may also include second internal threads 122 positioned at an outer bore region. The second internal threads 122 may be configured to engage with threads of the retaining ring 110.

The lower portion 104b may be positioned distally relative to the upper portion 104a when the outer screw body 102 may be in its collapsed configuration. The lower portion 104b may include internal threads 124 disposed within its inner bore. The internal threads 124 of the lower portion 104b may be configured to engage with corresponding threads on the inner bolt 106. The internal threads 124 of the lower portion 104b may have an opposite thread direction compared to the first internal threads 120 of the upper portion 104a.

The outer screw body 102 may include a non-threaded middle section 126 positioned between the upper portion 104a and the lower portion 104b. The non-threaded middle section 126 may facilitate alignment during insertion and may accommodate the axial separation of the upper and lower portions during expansion. The non-threaded middle section 126 may provide clearance for the movement of internal components during the expansion process.

The outer screw body 102 may be constructed from biocompatible materials suitable for permanent implantation within human tissue. The biocompatible materials may include titanium, stainless steel, or titanium alloys that may provide mechanical strength and corrosion resistance. The material selection may ensure long-term compatibility with the biological environment and may minimize adverse tissue reactions.

The outer screw body 102 may have a tapered configuration with a larger diameter at the proximal end and a smaller diameter at the distal end. The tapered configuration may facilitate insertion through the pedicle anatomy and may optimize the distribution of mechanical forces during expansion. The diameter variation may accommodate the anatomical constraints of the pedicle while providing adequate structural strength for load-bearing applications.

The outer screw body 102 may include surface treatments or coatings to enhance osteointegration and bone ingrowth. The surface treatments may include hydroxyapatite coatings, plasma spray applications, or acid etching to create micro-roughened surfaces. The enhanced surface characteristics may promote cellular attachment and may encourage bone formation around the implant interface.

### B. An Inner Bolt

The screw implant device 100 may include an inner bolt 106. The inner bolt 106 may comprise a threaded rod that may define the longitudinal axis of the expandable screw system. The inner bolt may be configured to engage with internal threads of both the upper portion 104a and the lower portion 104b of the outer screw body 102. The threaded engagement may enable controlled axial movement of the screw body portions 104a, 104b when the inner bolt 106 is rotated about its longitudinal axis.

The inner bolt 106 may include an upper threaded portion 106b that may engage with first internal threads of the upper portion 104a. The upper threaded portion may comprise a first thread direction that may provide mechanical engagement with corresponding internal threads within the upper portion of the outer screw body. The threading configuration may be designed to provide secure engagement while allowing controlled movement during expansion and/or retraction operations.

The inner bolt 106 may also include a lower threaded portion 106a that may engage with internal threads of the lower portion 104b. The lower threaded portion may comprise a second thread direction that may be opposite to the first thread direction of the upper threaded portion. The opposing thread configuration may enable simultaneous movement of both the upper and lower portions when the inner bolt is rotated in a single direction about its longitudinal axis.

An unthreaded middle portion 108 may be positioned between the upper threaded portion 106b and the lower threaded portion 106a of the inner bolt 106. The unthreaded middle portion may facilitate proper alignment of the threaded sections during assembly and operation. The middle portion 108 may also provide a transition zone between the opposing thread directions and may help maintain structural integrity of the inner bolt 106 during expansion and/or retraction operations.

The inner bolt 106 may include a tool interface 106c positioned at a proximal end of the upper threaded portion. The tool interface may be configured to engage with a second rotation tool for actuating the expansion mechanism. The tool interface may comprise a hexagonal socket, square drive, or other standardized connection that may enable secure engagement with surgical instrumentation. The tool interface may be accessible through the outer screw body to allow for intraoperative manipulation of the expansion mechanism.

The inner bolt 106 may be constructed from biocompatible materials suitable for permanent implantation. The material selection may include titanium, stainless steel, or titanium alloys that may provide the necessary mechanical strength for load-bearing applications. The biocompatible construction may ensure long-term compatibility with human tissue and may minimize the risk of adverse reactions or implant rejection.

The threading configuration of the inner bolt 106 may be designed to provide mechanical advantage during expansion and/or retraction operations. The thread pitch and profile may be selected to enable controlled movement while maintaining secure engagement with the outer screw body portions. The threading may also be configured to resist unwanted movement once the desired expansion may be achieved and the locking mechanism may be engaged.

The inner bolt 106 may be dimensioned to fit within the internal bore of the outer screw body 102 while maintaining adequate clearance for smooth operation. The diameter and length of the inner bolt may be optimized for the specific application and may vary based on the size of the outer screw body and the required expansion range. The dimensional relationships may ensure proper mechanical function while maintaining structural integrity of the overall implant system.

### C. An Expansion/Retraction Mechanism

The screw implant device 100 may include an expansion/retraction mechanism. As discussed above, the expansion/retraction mechanism may operate through a coordinated system of threaded components that enable controlled axial movement of the screw body portions 104a, 104b. The inner bolt 106 may comprise an upper threaded portion 106b and a lower threaded portion 106a that may engage with corresponding internal threads within the upper portion 104a and lower portion 104b respectively. The upper threaded portion 106b may have a first thread direction while the lower threaded portion 106a may have an opposite thread direction to enable simultaneous movement of both portions when the inner bolt 106 is rotated.

The threading configuration may provide mechanical advantage during the expansion and/or retraction processes through the opposing thread arrangement. As an example, when the inner bolt 106 is rotated in a clockwise direction, the upper threaded portion 106b may cause the upper portion 104a to move proximally while the lower threaded portion 106a may cause the lower portion 104b to move distally. This coordinated movement may result in axial separation of the screw body portions and overall lengthening of the implant.

The unthreaded middle portion 108 may be positioned between the upper threaded portion 106b and lower threaded portion 106a to provide proper alignment during the expansion and/or retraction process. The middle portion 108 may facilitate smooth operation of the threading mechanism and may prevent binding or misalignment of the threaded components during rotation. The length of the unthreaded middle portion 108 may be configured to accommodate the maximum expansion range of the implant.

The retaining ring 110 may provide a locking mechanism to secure the expanded configuration once the desired canal enlargement may be achieved. The retaining ring 110 may comprise external threads that may engage with second internal threads 122 of the upper portion 104a at an outer bore region. The retaining ring 110 may be rotated to advance along the second internal threads 122 until it may contact the inner bolt 106 or another component to prevent further rotation of the expansion mechanism.

The tool interface system may enable sequential manipulation of the expansion components through multiple access points. The first tool interface 118 may be positioned on the head 116 of the upper portion 104a to enable initial screw insertion using a first rotation tool. The second tool interface 106c may be located on a proximal end of the inner bolt 106 to enable expansion actuation using a second rotation tool. A third tool interface may be positioned on a proximal end of the retaining ring 110 to enable locking mechanism engagement using a third rotation tool.

The external threads 114 of the outer screw body 102 may provide secure anchoring within the vertebral anatomy during the expansion process. The external threads 114 may be configured to resist pullout forces generated during lamina elevation and may distribute mechanical loads across the pedicle and vertebral body structures. The thread pitch and profile may be optimized for bone purchase while allowing for controlled insertion through standard surgical techniques.

The expansion mechanism may be configured to provide controlled force application during lamina elevation. The mechanical advantage provided by the threading arrangement may enable surgeons to apply precise amounts of distraction force based on patient anatomy and pathology. The expansion rate may be controlled through the rotational input applied to the inner bolt 106, allowing for gradual lamina elevation and real-time assessment of neural decompression.

The biocompatible construction of the expansion mechanism components may ensure long-term functionality within the biological environment. The inner bolt 106 and outer screw body 102 may be manufactured from titanium alloys or stainless steel to provide corrosion resistance and mechanical durability. The threading surfaces may be precision machined to ensure smooth operation and reliable engagement throughout the expansion process.

The reversible nature of the expansion mechanism may allow for intraoperative adjustment of the canal enlargement. The inner bolt 106 may be rotated in the opposite direction to reduce the expansion if excessive distraction may be achieved. This reversibility feature may provide surgeons with precise control over the final canal diameter and may enable optimization of the decompression based on patient response and anatomical considerations.

### D. A Retaining Ring

In some embodiments, the screw implant device 100 may include aretaining ring 110. The retaining ring 110 may comprise a cylindrical component configured to provide a secure locking mechanism for the expandable screw system. The retaining ring 110 may be positioned at a proximal end of the outer screw body 102 and may be configured to engage with the upper portion 104a through a threaded interface. The retaining ring 110 may include external threads 112 disposed along its outer circumference that may be configured to mate with second internal threads 122 of the upper portion 104a.

The external threads 112 of the retaining ring 110 may have a thread pitch and profile that may be compatible with the second internal threads 122 of the upper portion 104a. The threading engagement may enable the retaining ring 110 to be advanced along the second internal threads 122 through rotational motion. The retaining ring 110 may be rotated in a clockwise direction to advance distally along the threads or in a counterclockwise direction to retract proximally along the threads.

The retaining ring 110 may include a tool interface 113 positioned at its proximal end to enable engagement with a third rotation tool. The tool interface 113 may comprise a hexagonal socket, square drive, or other standardized connection that may allow for secure engagement with surgical instrumentation. The tool interface 113 may be accessible from the proximal end of the expandable screw system to allow for intraoperative manipulation of the locking mechanism.

The retaining ring 110 may be configured to contact the inner bolt 106 when advanced to its locking position. The distal end of the retaining ring 110 may include a contact surface 115 that may engage with a proximal surface of the inner bolt 106 to prevent further rotation of the expansion mechanism. The contact surface 115 may be configured to distribute the locking force evenly across the inner bolt 106 to prevent damage to the threading or other components.

The retaining ring 110 may be constructed from biocompatible materials suitable for permanent implantation within human tissue. The material selection may include titanium, stainless steel, or titanium alloys that may provide mechanical strength and corrosion resistance. The biocompatible construction may ensure long-term compatibility with the biological environment and may minimize adverse tissue reactions.

The retaining ring 110 may include a flange portion 117 that may extend radially outward from the main body of the ring. The flange portion 117 may provide additional surface area for tool engagement and may help distribute mechanical loads during the locking process. The flange portion 117 may also serve as a visual indicator of the retaining ring position during surgical procedures.

The internal bore 119 of the retaining ring 110 may be dimensioned to provide clearance for the inner bolt 106 while maintaining adequate wall thickness for structural integrity. The internal bore 119 may include a smooth finish to minimize friction during assembly and operation. The bore diameter may be selected to accommodate the maximum diameter of the inner bolt 106 while providing sufficient clearance for smooth operation.

The retaining ring 110 may include anti-rotation features 121 that may prevent unwanted loosening once the locking position may be achieved. The anti-rotation features 121 may comprise thread-locking compounds, mechanical detents, or surface treatments that may increase the resistance to rotational movement. These features may ensure that the expanded configuration may be maintained throughout the healing process without requiring additional intervention.

### III. Platform Operation

The method stages described herein may be performed by one or more actors, including but not limited to surgeons, surgical assistants, medical technicians, or automated systems. The various stages may be carried out by a single actor or may be distributed among multiple actors working in coordination. The order of the stages may be modified, rearranged, or performed in alternative sequences based on surgical requirements, patient anatomy, or clinical judgment. Certain stages may be performed simultaneously or in overlapping timeframes rather than in strict sequential order. Additional stages may be inserted between the disclosed stages, and certain stages may be omitted or combined with other stages depending on the specific surgical approach or patient needs.

The method may be adapted to accommodate different surgical techniques, anatomical variations, or equipment configurations without departing from the scope of the disclosure. The stages may be performed using different instruments, tools, or approaches than those specifically described, provided that the functional objectives of each stage may be achieved. The method may be scaled for single-level or multi-level procedures, with stages repeated or modified as appropriate for the specific surgical scope.

The temporal relationships between stages may be adjusted based on intraoperative findings, patient response, or surgical judgment. Certain stages may be performed in preparation phases prior to the main surgical procedure, while other stages may be performed during post-operative monitoring or follow-up periods. The method may incorporate decision points where the surgical approach may be modified based on real-time assessment of anatomical conditions or procedural outcomes.

The method may be performed using minimally invasive techniques, open surgical approaches, or hybrid approaches combining elements of both. The stages may be adapted for percutaneous procedures, endoscopic procedures, or robot-assisted procedures depending on the available technology and surgeon preference. The method may incorporate imaging guidance systems, navigation systems, or other technological aids to enhance precision and safety during stage execution.

Also disclosed herein are methods of making and using the disclosed screw implants, devices, apparatuses, and systems. For example, other exemplary aspects, techniques of the present disclosure provide a method of performing spinal surgery, including: inserting a screw implant into a vertebra, the screw implant having an internal expansion mechanism; actuating the internal expansion mechanism to expand the screw implant from a first length to a second length greater than the first length; and locking the screw implant at the second length.

In some aspects, the techniques described herein relate to a method of performing spinal surgery, including: inserting a screw implant into a vertebra, the screw implant having a proximal portion, a distal portion, and an internal expansion mechanism connecting the proximal portion and the distal portion; actuating the internal expansion mechanism to axially separate the proximal portion from the distal portion; and locking the proximal portion and the distal portion in the axially separated position. In some embodiments, the implant can be used for compression. In such case, the movable portion would be operated to move in the opposite direction for use cases where compression is needed. For example, the implant may be inserted already expanded, then the internal mechanism can function in the closing direction in different applications, such as, for example, for closing fractures in different bones, and the like.

In some aspects, the techniques described herein relate to a method of expanding a spinal canal, including: inserting a screw body into a vertebral pedicle; actuating an internal mechanism within the screw body to increase a length of the screw body, thereby elevating a portion of the vertebra; and engaging a locking mechanism to maintain the increased length of the screw body.

In some aspects, the techniques described herein relate to a method for expanding a spinal canal, the method including: inserting a pedicle screw through a pedicle of a vertebra, the pedicle screw having an elongated body with an internal mechanism; actuating the internal mechanism to cause linear translation of at least a portion of the elongated body; and engaging a locking mechanism to maintain the linear translation of the portion of the elongated body.

In some aspects, the techniques described herein relate to a method of expanding a spinal canal, including: creating a pedicle passage in a vertebra; inserting an expandable pedicle screw through the pedicle passage and into a vertebral body of the vertebra; expanding the expandable pedicle screw to lengthen the pedicle passage; and elevating a lamina of the vertebra using the expanded pedicle screw to increase a diameter of the spinal canal.

In some aspects, the techniques described herein relate to a method of treating spinal stenosis, including: accessing a pedicle of a vertebra; creating a pedicle passage through the pedicle and into a vertebral body of the vertebra; inserting an expandable pedicle screw through the pedicle passage; expanding the expandable pedicle screw to lengthen the pedicle; and increasing a cross-sectional area of a spinal canal adjacent to the vertebra by elevating a lamina of the vertebra using the expanded pedicle screw.

In some aspects, the techniques described herein relate to a method of performing a thoracolumbar laminoplasty, including: accessing a thoracolumbar spine of a patient; identifying a target vertebral level; inserting a laminoplasty screw through a lamina, pedicle, and into a vertebral body of a vertebra at the target vertebral level; disconnecting a proximal portion of the pedicle; and elevating the lamina using the inserted laminoplasty screw to increase a diameter of a central spinal canal.

In some aspects, the techniques described herein relate to a method for expanding a spinal canal, including: inserting an outer sleeve of a pedicle-lengthening screw implant through a pedicle of a vertebra; rotating an inner bolt disposed within the outer sleeve to cause an upper portion and a lower portion of the outer sleeve to separate; and radially extending expandable flanges connected to the lower portion of the outer sleeve into a widened pedicle cut as the upper and lower portions separate to expand the spinal canal.

In some aspects, the techniques described herein relate to a method for expanding a spinal canal, including: inserting a pedicle-lengthening screw implant through a pedicle of a vertebra; actuating an inner expansion mechanism of the pedicle-lengthening screw implant; and radially extending expandable elements of the pedicle-lengthening screw implant into a widened pedicle cut to expand the spinal canal.

In some aspects, the techniques described herein relate to a method of expanding a spinal canal using a pedicle-lengthening screw implant, including: inserting an outer sleeve of the pedicle-lengthening screw implant through a pedicle of a vertebra, the outer sleeve having an upper portion and a lower portion; rotating an inner bolt disposed within the outer sleeve to cause the upper and lower portions of the outer sleeve to separate; and radially extending one or more expandable flanges connected to the lower portion of the outer sleeve in response to the separation of the upper and lower portions.

In some aspects, the techniques described herein relate to a method for expanding a spinal canal, including: inserting a pedicle-lengthening screw implant through a pedicle of a vertebra; activating an expansion mechanism integrated within the pedicle-lengthening screw implant; and radially extending expandable portions formed on a distal section of the pedicle-lengthening screw implant to widen a pedicle cut and expand the spinal canal.

### A. Master Method

### Screw Implant and System Operation and Methods for Using Same

Consistent with embodiments of the present disclosure, a method may be performed by at least one of the aforementioned components.

A surgical procedure may be initiated, and a structural tissue channel may be established within the vertebral anatomy. This may involve creating a pedicle passage through drilling techniques and may include the use of specialized instruments such as a pedicle drill 902 and pedicle cutting tool 904.

The method may continue with insertion of the screw implant device 100 into the established structural tissue channel in its collapsed configuration. The outer screw body 102 may be advanced through the pedicle passage using standard insertion techniques. The collapsed configuration may facilitate insertion through the anatomical constraints of the pedicle while maintaining the structural integrity of the surrounding bone tissue.

The surgeon may verify proper positioning and orientation of the screw implant within the vertebral anatomy. This verification stage may utilize imaging guidance systems such as fluoroscopy or computed tomographic imaging to confirm accurate placement. The positioning verification may ensure that the screw implant may be optimally located for effective expansion and lamina elevation.

The inner bolt 106 may be rotated until the desired expanded configuration may be achieved. The rotation of the inner bolt 106 may cause the upper portion 104a and lower portion 104b of the outer screw body 102 to separate axially along the longitudinal axis. This axial separation may create the mechanical force necessary to elevate the lamina and expand the spinal canal diameter. The rotation process may be controlled through the second tool interface 106c positioned on the proximal end of the inner bolt 106.

Instrumentation may be attached to the screw implant assembly to secure the expanded configuration. This stage may include the engagement of the retaining ring 110 through its external threads 112 with the second internal threads 122 of the upper portion 104a. The locking mechanism may prevent unwanted rotation of the inner bolt 106 and may maintain the expanded position permanently throughout the healing process.

Upon completion of the surgical procedure, the expanded screw implant may provide sustained spinal canal enlargement while maintaining the structural integrity of the vertebral anatomy. The minimally invasive nature of the procedure may result in reduced surgical trauma compared to traditional laminectomy approaches.

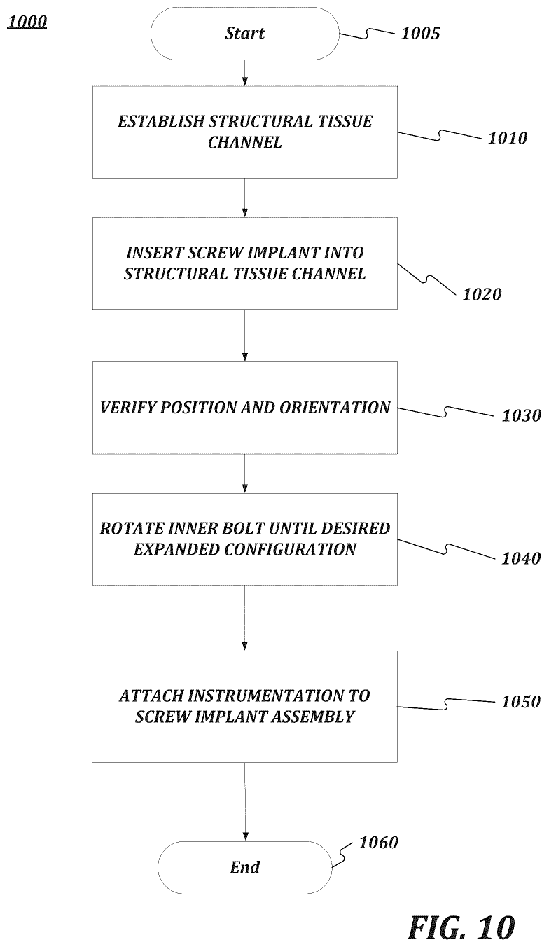

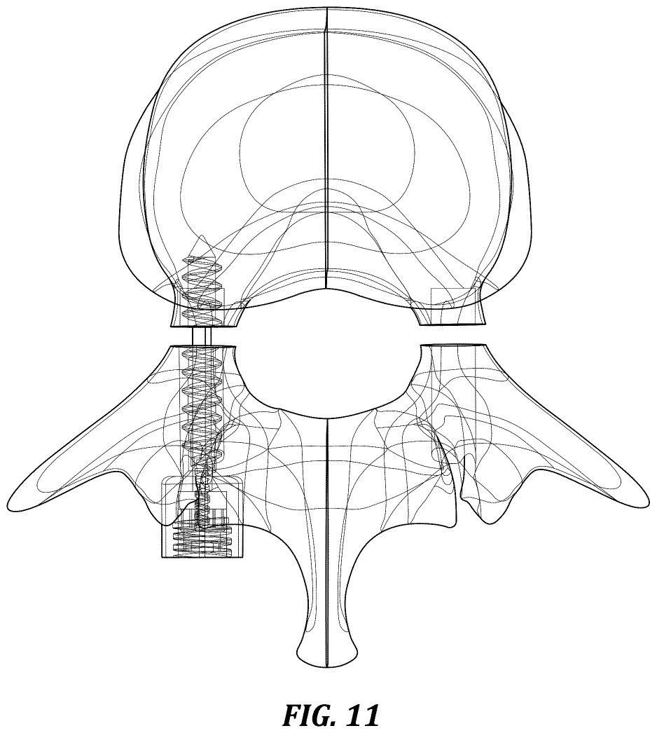

FIG. 10 is a flow chart is depiction setting forth various stages involved in a method 1000 consistent with an embodiment of the disclosure for using the disclosed screw implants, apparatuses, devices, and systems. Method 1000 may be implemented using, at least in part, for example, devices, as described in more detail with respect to FIGS. 1A-9.

Screw implant device 100 may comprise outer screw body 102 and inner shaft 106 for operating the device components, for example, for axially separating upper and lower portions 104 from a closed (or collapsed) configuration to an open (or expanded) configuration upon rotation of the inner shaft within the screw body. Furthermore, although stages are disclosed with reference to device 100, it should be understood that other disclosed device embodiments may enable the operation of method 1000, including, but not limited to, other device mechanisms, mechanical components, tissue properties (e.g., bone type, etc.), patient conditions, and the like. Further still, although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein.

It will be appreciated that screw implants and devices as presented herein may be useful in a variety of surgical procedures, e.g., spinal surgery, elevation of the thoracolumbar lamina, laminoplasty, and the like. One advantage of the present invention is that it may be suitable for use with existing surgical techniques procedures requiring expansion of the spinal canal. In relevant part, implanting screw implants according to the present disclosure may involve a surgeon creating a channel in a patient's bone, e.g., within a pedicle and/or an associated vertebral body. In one aspect, a method for treating spinal stenosis is provided herein where a spinal canal is enlarged by cutting a vertebra through one or both pedicles, separating the vertebral cut and then stabilizing the cut, allowing the vertebra to heal with the spinal canal expanded, permanently creating more space for the spinal nerves, thus relieving compression on the nerves. In another aspect, the method of expanding the spinal canal includes drilling a passage or hollow tunnel into one or both pedicles of a vertebra, making a pedicle cut (or osteotomy) from within the passage through to the spinal canal and to the outside of the vertebra, distracting (elongating) the osteotomy to expand the spinal canal, and then stabilizing the osteotomy. In another aspect, the method of expanding the spinal canal includes the following steps: first, a guide wire is inserted into a central portion of the vertebral pedicles on each side of a vertebra. This and other method steps can be accomplished with the assistance of imaging techniques such as x-rays, fluoroscopy, or computer assisted image guidance technology.

Next, the guide wire is used to direct the position of a cannulated drill (drill with a central barrel or passage to allow introduction over the guide wire) into each of pedicles to form a passage or hollow tunnel in the central portion of each pedicle. After this stage, the pedicles comprise a hollow column of bone having a central passage and thin, cylindrical, bony walls.

Next, the vertebral pedicles are cut circumferentially, forming an upper portion and a lower portion. A cutting tool, such as the pedicle cutting tools and saws disclosed in the disclosure, can be introduced into the central passage in each pedicle to perform the circumferential cut. The cutting tool has a cutting surface that projects radially outward so that the bony walls of each pedicle can be circumferentially cut. With both pedicles circumferentially cut, the vertebra is divided into an upper portion (including the spinous process, lamina, transverse process and articular processes) and a lower portion (including the vertebral body). As disclosed herein, the cutting tool could include a rotating cutting burr or osteotome (chisel) as the cutting surface, both of which are well known in the art.

Finally, each osteotomy (site of the circumferential bone cut) is distracted (expanded). A specially designed implant, such as those of the present invention, can be used to distract the osteotomy. Generally, the implants of the present invention include an upper portion, a lower portion, and an inner member communicating and interacting with the upper and lower portion to distract the upper portion from the lower portion, thereby separating the vertebral cut and expanding the spinal canal.

A next step may involve the surgeon inserting the entire screw implant, in the collapsed configuration, into the channel. Once properly positioned in the desired location, the surgeon may then rotate the inner bolt or shaft within the screw body channel, e.g., by inserting a rotation tool into the channel and turning the inner bolt such that the bolt threads engage the inner threads of the support channel. Rotating the inner bolt within the channel may cause one or more movable portions of the screw implant to move axially. The surgeon may continue to rotate the inner bolt until the desired amount of lengthening of the screw implant has been achieved.

In stage 1010, a passage or channel is established or formed in a portion of structural tissue, such as bone. The formation of the channel may be accomplished by various surgical methods based on the desired anatomical location and patient-specific factors. For example, a cylindrical passage may be created through the pedicle of a vertebra using a cannulated drill, which may be guided over a guidewire for accuracy. Once the initial channel is created, a specialized pedicle cutting tool may be introduced to perform a circumferential osteotomy around the central passage and create a separation between upper and lower portions of the pedicle. The cutting tool may include flexible elements configured to conform to the anatomy and perform controlled, circumferential bone cuts. The depth and position of the cuts may be adjusted intraoperatively depending on the anatomical constraints and therapeutic objective. In some embodiments, the channel may also be established in non-bone structural tissues or at alternate vertebral sites, depending on the clinical application and requirements.

By way of example, in one embodiment, stage 1010 may involve forming a passage through both pedicles of a lumbar vertebra using fluoroscopic guidance to carefully align the trajectory with the vertebral axis. After confirming the position with imaging, a cannulated drill may be advanced to create the central passage, and a rotary cutting burr may then form a precise circumferential cut within the pedicle. In another embodiment, the channel may be formed using a minimally invasive approach, where a guidewire is inserted percutaneously under imaging guidance, followed by sequential dilation to accommodate the cutting tool. In some circumstances, only one pedicle may be treated, or a partial channel may be established if full circumferential access is not required. An additional embodiment may apply the process to thoracic or sacral vertebrae, where the anatomy and access considerations may dictate alternative orientations or instrument sizes. These variations enable surgeons to adapt the technique of stage 1010 to a wide range of anatomical sites and patient needs.

In stage 1020, a screw implant in a closed or collapsed configuration may be inserted into the previously formed structural tissue channel. The screw implant may comprise an outer screw body and an inner bolt configured to enable axial expansion after placement. The insertion may utilize fluoroscopic or other imaging guidance to ensure accurate trajectory through the established channel. During placement, the orientation and depth of insertion may be determined based on anatomical landmarks, preoperative planning, and the intended therapeutic outcome. The screw implant may be advanced using a compatible insertion tool that interfaces with the proximal head of the implant. The design of the implant may facilitate engagement with bone threads for optimal fixation.

For example, in one embodiment, the screw implant may be inserted through a cylindrical passage formed in the pedicle of a lumbar vertebra, with the trajectory confirmed using lateral and anteroposterior fluoroscopic views. The surgeon may use a cannulated driver over a guidewire to guide the screw into position before removing the guidewire and confirming implant placement with imaging. In another embodiment, insertion may be performed during a minimally invasive procedure, in which the screw is delivered through a small percutaneous incision using sleeve-based guides. The screw may be left in its collapsed state at this stage, enabling subsequent expansion only after final placement is confirmed. In an alternative example, the implant may be designed with expandable flanges or radially extendable blades, which remain housed within the screw body during insertion to reduce channel size requirements and minimize tissue disruption. Following placement, the expansion mechanism may be activated in subsequent stages, allowing for a wide range of anatomical and patient-specific use scenarios.

In stage 1030, the proper positioning and orientation of the screw implant may be verified using visual cues, radiopaque markers, and imaging techniques such as fluoroscopy or radiography. Markers associated with the screw implant may aid in confirming its location within the channel or pedicle, the entry angle, the depth of insertion, and the relationship of the implant to surrounding anatomical structures. Verification may include assessment of the length and width of the screw, confirmation that the implant trajectory corresponds to the planned orientation, and evaluation of whether the implant has reached the intended depth or vertebral segment. Intraoperative imaging may inform immediate adjustments before proceeding to expansion or further manipulation.

As one example, in an embodiment, after insertion of the screw implant into the pedicle, the surgeon may acquire anteroposterior and lateral fluoroscopic images to verify the trajectory and depth. Radiopaque features on the screw head and along the shaft may help confirm that the screw follows the intended anatomical path and is not in proximity to neural structures. In another embodiment, the system may include mechanical or digital depth markers on the insertion tool that indicate the position of the screw as it is advanced, providing direct feedback to the surgeon. In minimally invasive approaches, intraoperative navigation systems or optical tracking may be employed to provide three-dimensional visualization and positioning guidance relative to preoperative imaging data. Additional embodiments may employ sensors or indicators integrated with the implant or tools to provide real-time feedback on orientation or contact with bone boundaries. These features enable verification in a range of clinical settings, supporting both conventional and advanced imaging or navigation modalities to ensure accurate placement before implant expansion.

In stage 1040, the inner bolt or shaft of the screw implant may be rotated within the screw body, causing the upper and lower portions of the implant to separate axially and lengthen the device from its collapsed configuration to an expanded configuration. This expansion may generate a distraction force across the osteotomy or cut site, effectively separating the upper bony segment (such as the lamina) from the lower segment (such as the vertebral body) and thereby enlarging the spinal canal. The amount of expansion may be determined intraoperatively based on anatomical landmarks and imaging feedback, allowing for controlled and gradual canal decompression. The expansion mechanism may permit fine-tuning to optimize therapeutic benefit while minimizing risk to adjacent neural and support structures. Throughout this process, the screw implant maintains engagement with bone threads to provide stability during and after distraction.

For example, in a representative embodiment, after verifying implant position with imaging, the surgeon may engage the expansion tool with a tool interface at the proximal end of the inner bolt and apply controlled rotational input. As the inner bolt rotates, the threaded interfaces of the outer screw body translate the rotation into axial movement, separating the upper and lower portions of the implant. This results in progressive elevation of the lamina, visualized intraoperatively by an increase in the spinal canal diameter. In another embodiment, the expansion may be performed incrementally, with the surgeon intermittently checking neural decompression and stopping when the desired anatomical space is achieved. Alternatively, the system may feature mechanical stops or feedback indicators, such as torque-limiting tools or audible clicks, to guide the surgeon on expansion limits. Some versions of the implant may allow for reversible expansion, enabling the device to be partially collapsed if excessive distraction is observed during imaging or physical assessment. These variations allow adaptation to various surgical scenarios, anatomical variations, and patient-specific therapeutic requirements.

In stage 1050, the locking mechanism of the screw implant may be engaged to maintain the expanded configuration achieved in the previous stage. The locking mechanism may include a retaining ring, threaded cap, ratcheting assembly, or cam-actuated system that secures the inner bolt and prevents further axial movement of the upper and lower portions of the screw body. Engagement of the locking mechanism may involve rotating a proximal tool interface with a dedicated instrument until tactile or audible feedback indicates secure locking. This step ensures the expanded screw implant remains stable, resists collapse or migration, and maintains distraction across the osteotomy for the duration of post-operative healing. The type of locking mechanism and the method of engagement may vary based on the implant design and surgical preferences.

For example, in one embodiment, a retaining ring may be advanced along internal threads at the proximal end of the screw body by rotating it with a hex or torque-limiting instrument until it contacts the inner bolt, thus preventing further rotation or retraction. In another embodiment, the locking may be accomplished via a ratcheting mechanism that automatically engages as expansion is performed, requiring a specific unlocking tool or sequence for future revision. Alternatively, a cam-based system may enable the surgeon to rotate the locking element into place, with a visual indicator confirming secure engagement. Some designs may also incorporate mechanical detents or thread-locking compounds to resist inadvertent loosening during physiological loading. These locking approaches enable surgeons to select the preferred mechanism based on anatomical challenges, implant accessibility, and anticipated post-operative stability needs.

Post-expansion assessment and final verification of spinal canal decompression may be performed. This stage may involve intraoperative imaging, such as fluoroscopy or computed tomography, to confirm that the desired anatomical changes have been achieved and that the spinal canal has been adequately enlarged. The position and stability of the expanded and locked screw implant may be evaluated for any signs of migration or hardware compromise. The surrounding neural and bony structures may be inspected visually if direct access is available, or through image-guided navigation if a minimally invasive technique is employed. Following successful verification, surgical instruments and retractors may be removed, and the operative site may be irrigated and closed according to standard procedures. The patient may then be monitored throughout the postoperative period with serial imaging to confirm long-term maintenance of decompression and hardware stability.

For example, in one embodiment, after engaging the locking mechanism and achieving the planned expansion, intraoperative lateral and anteroposterior fluoroscopic images may be obtained to confirm the increase in spinal canal diameter and the final construct's integrity. In another embodiment, intraoperative neurophysiological monitoring may be used to assess neural function and ensure that decompression has been effective without causing iatrogenic injury. Alternatively, the surgeon may use three-dimensional intraoperative CT scans or navigation-assisted systems to obtain detailed confirmation of implant position relative to anatomical landmarks and neural structures. Postoperative monitoring may include scheduled radiographs or CT studies to evaluate the long-term maintenance of vertebral alignment and hardware position, and to assess for potential complications such as loosening or subsidence.

Finally, the method 1000 may end at stage 1060.

Also disclosed herein are kits comprising the disclosed screw implant and devices. For example, in an exemplary aspect, the present disclosure provides a pedicle screw kit comprising: a disclosed screw implant or device; and instructions for using the device in connection with a medical procedure. In further aspects, the instruction and/or method may comprise any surgical method and/or method step. In still further aspects, the method and/or method step may comprise affixing or implanting a disclosed screw implant or device to a patient, such as a human or mammal.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. A pedicle-lengthening screw implant for expanding a spinal canal, comprising:
an outer screw body configured to be inserted through a pedicle of a vertebra, the outer screw body having an upper portion and a lower portion; and
an inner bolt disposed within the outer screw body, defining a longitudinal axis and configured to interact with each of the upper and the lower portions;
wherein rotation of the inner bolt about the longitudinal axis causes the upper and lower portions of the outer screw body to separate axially along the longitudinal axis.

2. The pedicle-lengthening screw implant of claim 1, wherein the outer screw body comprises external threads configured to engage bone of the pedicle.

3. The pedicle-lengthening screw implant of claim 1, wherein the inner bolt comprises a threaded rod that engages first internal threads of the upper portion and internal threads of the lower portion at an inner bore of the outer screw body.

4. The pedicle-lengthening screw implant of claim 3, wherein the first internal threads of the upper portion and the internal threads of the lower portion have opposite thread directions.

5. The pedicle-lengthening screw implant of claim 1, wherein the inner bolt comprises an upper portion with a first thread direction and a lower portion with an opposite thread direction.

6. The pedicle-lengthening screw implant of claim 1, wherein the inner bolt comprises an unthreaded middle portion between a threaded upper portion and threaded lower portion.

7. The pedicle-lengthening screw implant of claim 1, further comprising a retaining ring configured to secure the position of the inner bolt and prevent rotation thereof, wherein the retaining ring comprises external threads that engage second internal threads of the upper portion at an outer bore of the outer screw body.

8. The pedicle-lengthening screw implant of claim 1, wherein the outer screw body comprises a head at a proximal end of the upper portion, the head having a first tool interface configured to engage a first rotation tool.

9. The pedicle-lengthening screw implant of claim 1, further comprising a second tool interface at a proximal end of the inner bolt, the second tool interface being configured to engage a second rotation tool.

10. The pedicle-lengthening screw implant of claim 7, further comprising a third tool interface at a proximal end of the retaining ring, the third tool interface being configured to engage a third rotation tool.

11. The pedicle-lengthening screw implant of claim 1, wherein the outer screw body comprises a biocompatible metal.

12. The pedicle-lengthening screw implant of claim 1, wherein the inner bolt comprises a biocompatible metal.

13. A screw implant for spinal surgery, comprising:
a screw body configured for insertion into a vertebra;
an internal mechanism disposed within the screw body; and
an actuator configured to operate the internal mechanism,
wherein operation of the internal mechanism causes the screw body to expand from a first length to a second length greater than the first length.

14. The screw implant of claim 13, wherein the screw body comprises:
a proximal portion having external threads; and
a distal portion having external threads,
wherein operation of the internal mechanism causes the proximal portion and distal portion to separate axially.

15. The screw implant of claim 14, wherein the proximal portion and distal portion are connected by a telescoping mechanism.

16. The screw implant of claim 13, wherein the internal mechanism comprises a threaded rod.

17. The screw implant of claim 16, wherein rotation of the threaded rod causes axial expansion of the screw body.

18. The screw implant of claim 13, further comprising a locking mechanism configured to maintain the screw body at the second length.

19. The screw implant of claim 13, wherein the actuator comprises a tool interface at a proximal end of the screw body.

20. The screw implant of claim 13, wherein the internal mechanism is reversible to allow the screw body to move from the second length to the first length.
